(19) United States
(12) Patent Application Publication
(10) Pub. No.: US 2024/0079085 A1
(43) Pub. Date: Mar. 7, 2024

(54) MACHINE-LEARNED FEATURE CORRELATIONS FROM HIGH FEATURE DENSITY BIOLOGICAL IMAGES

(71) Applicant: Spring Discovery, Inc., San Carlos, CA (US)

(72) Inventors: Charles Reid Marsh, Brooklyn, NY (US); Lauren Nicolaisen, Redwood City, CA (US); Colin Fuller, Montreal (CA); Brandon White, San Francisco, CA (US); Benyamin Komalo, San Francisco, CA (US)

(21) Appl. No.: 17/903,932

(22) Filed: Sep. 6, 2022

(51) Int. Cl. G16B 15/30; G16B 40/00; G16B 50/30

(52) U.S. Cl. CPC G16B 15/30; G16B 40/00; G16B 50/30

(57) ABSTRACT

A high density correlation system can train a machine-learned model to determine one or more phenotypes of a cell and identify compounds corresponding to a user-queried phenotype. The high density correlation system can generate training data using single-cell images and train the machine-learned model using the generated training data. The machine-learned model can determine phenotypes of cells based on images of the cells. The high density correlation system can generate a database that includes phenotype-compound mappings generated based on the outputs of the machine-learned model. After receiving a query that identifies a phenotype, the high density correlation system can generate a result set of the query using the database for display at a graphical user interface (GUI). The result set can identify compounds corresponding to the identified phenotype. Additionally, the displayed compounds can be ordered based on a score for each compound.

This disclosure relates generally to cell screening, and more specifically to machine-learned phenotypic profiling in cell screening.

### BACKGROUND

Cell screening can produce hundreds of terabytes of data. Vast numbers of studies are performed to understand the effect of drugs on immune cells stimulated by thousands of conditions. A massive number of images are produced from these studies, and each image can depict hundreds of cell phenotypes. Scientists are tasked to process this sizeable quantity of phenotype data to identify new treatments for various conditions. While scientists can turn to computing to help process these images, conventional systems expend a considerable amount of processing resources to provide scientists with even a modicum of understanding of what might be uncoverable in a vast depth of high-dimensional cell data.

### SUMMARY

A system and method for processing and visualizing high-dimensional cell data is described herein. A high density correlation system applies machine learning to terabytes of cell imaging data and proteomic data to aid scientists in the drug discovery process. Using the high density correlation system, scientists can visually process vast amounts of data and use correlations determined by the system to understand connections between a large number of compounds. In some embodiments, without specifying to the high density correlation system what is being searched, the system can present identified correlations: connections between treatment conditions, relationships between donor populations, correlations between phenotypic features, and more.

In one embodiment, the high density correlation system can train a machine-learned model to determine one or more phenotypes of a cell and identify compounds corresponding to a user-queried phenotype. The high density correlation system can generate training data using single-cell images and train the machine-learned model using the generated training data. The machine-learned model can determine phenotypes of cells based on images of the cells. The high density correlation system can generate a database that includes phenotype-compound mappings generated based on the outputs of the machine-learned model. After receiving a query, from a client device, that identifies a phenotype, the high density correlation system can generate a result set of the query using the database for display at a graphical user interface (GUI). The result set can identify compounds corresponding to the identified phenotype. Additionally, the displayed compounds can be ordered based on a score for each compound.

### BRIEF DESCRIPTION OF THE DRAWINGS

FIG. 1 depicts a process for creating phenotypic fingerprints using a high density feature data, in accordance with at least one embodiment.

FIG. 2 is a block diagram of a system environment in which a high density correlation system operates, in accordance with at least one embodiment.

FIG. 3 shows a block diagram of a process for providing a visualization of phenotypic fingerprints of a biological sample, in accordance with at least one embodiment.

FIG. 4 shows a GUI for identifying compounds with customized scoring, in accordance with at least one embodiment.

FIG. 5 shows a GUI for identifying compounds that correlate with an effect on a condition, in accordance with at least one embodiment.

FIG. 6 shows a GUI for querying a high density correlation system, in accordance with at least one embodiment.

FIG. 7 shows a GUI for visualizing profiles of cells, in accordance with at least one embodiment.

FIG. 8 is a flowchart illustrating a process for identifying phenotypic fingerprints using the high density correlation system described herein, in accordance with at least one embodiment.

The figures depict various embodiments for purposes of illustration only. One skilled in the art will readily recognize from the following discussion that alternative embodiments of the structures and methods illustrated herein may be employed without departing from the principles described herein.

### DETAILED DESCRIPTION

### System Architecture

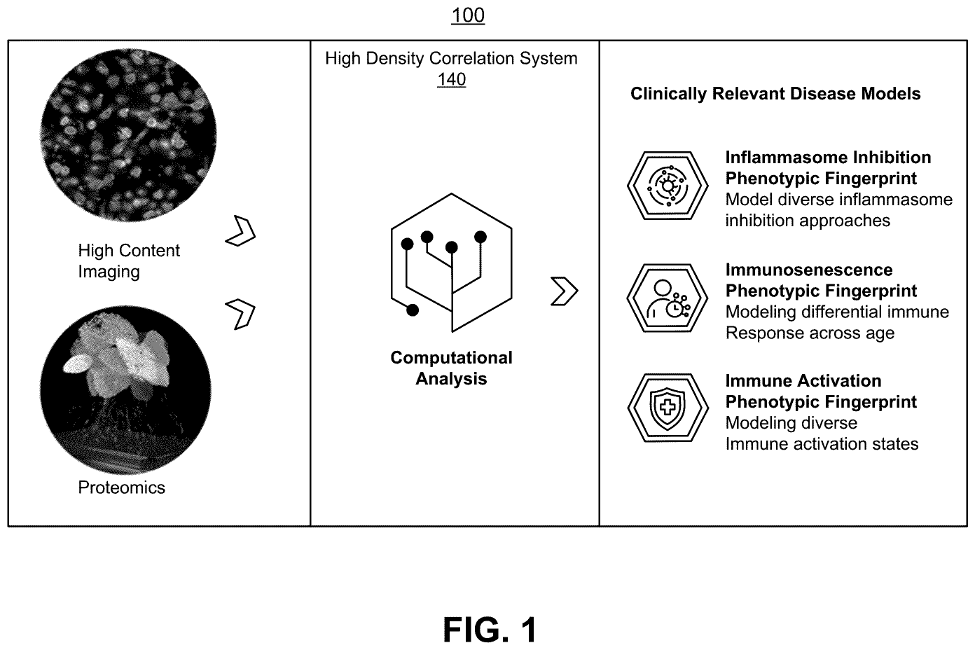

FIG. 1 depicts a process for creating phenotypic fingerprints using high density feature data, in accordance with at least one embodiment. As referred to herein, a phenotypic fingerprint is a quantitative representation using one or more phenotypes for identifying a biological condition (e.g., rheumatoid arthritis, psoriasis, eye diseases such as keratitis, etc.) or behavior (e.g., immune responses, inflammasome inhibition, etc.). A high density correlation system 140 performs computational analysis to generate phenotypic fingerprints for modeling cell phenotypes across various conditions (e.g., thousands of conditions) to discover compounds for treating diseases. Additionally, the high density correlation system 140 may provide an interactive interface to display cell phenotypes and correlations between those behaviors and compounds. The interactive interface may receive user input to query the phenotypic fingerprints generated by the high density correlation system 140 for information related to particular compounds, cell phenotypes, or conditions. In some embodiments, the high density correlation system 140 may receive terabytes of lab generated data (e.g., images of cells).

The high density correlation system 140 may apply a suite of machine learning models that measures hundreds of high-content imaging and proteomics features. The models enable the system 140 to build comprehensive cellular models of complex diseases with deeper understanding of drugs' on-target actions, off-target effects, and safety signals. Graphical user interfaces (GUIs) generated by the high density correlation system 140 may be interactive and unify cellular function, morphology, metabolomics, proteomics, spatial interactions, and more. Users may use the high density correlation system 140 to map hypotheses to specific, functional understanding provided by the machine learning models of the system 140. The high density correlation system 140 may receive samples of human immune cells (e.g., peripheral blood mononuclear cells (PBMC's)) stimulated by thousands of conditions, which results in hundreds of terabytes of imaging and proteomic data for input into one or more machine learning models of the system 140.

In one example, the high density correlation system can create an inflammasome activation phenotypic fingerprint and identify inflammasome inhibitors. The high density correlation system 140 receives high content imaging of PBMC samples dosed with known inflammasome control compounds and proteomics indicating the concentration of particular proteins over time. As referred to here, in the terms “dose” and “treat” may be used interchangeably unless otherwise apparent from the context in which they are used. The proteomics can include data showing the concentration of proteins activating inflammasomes. Such proteins can include anthracis toxin, muramyl dipeptide (MDP), damage-associated molecular patterns (DAMPs), pathogen associated molecular patterns (PAMPs), flagellin, or double-stranded DNA (dsDNA) binding proteins. Examples of inflammasomes can include the NLRP1 inflammasome, NLRP3 inflammasome, NLRC4 inflammasome, and the AIM2 inflammasome. The known inflammasome control compounds can include MCC950, disulfiram (DSF), Z-VAD, or intermedin (IMD). The high density correlation system 140 processes the high content imaging and the proteomics to generate fingerprints representative of cell phenotypes demonstrated in the high content images. The high density correlation system 140 can map each fingerprint to a compound applied to the cells to affect the represented phenotypes. The high density correlation system 140 can identify particular compounds, which are inflammasome inhibitors in this example, based on a user's desired effect of applying a particular compound. For example, a user can request that the high density correlation system show compounds that effected the particular phenotype of increasing macrophage polarization. The inflammasome activation phenotypic fingerprints and identified inflammasome inhibitors can be used to treat conditions and in particular, inflammation diseases such as rheumatoid arthritis, psoriasis, macrophage activation syndrome, or chronic kidney disease.

The high density correlation system 140 generates phenotypic fingerprints based on at least images of cells depicting a high density of information or content describing phenotypes of the cells. The high density correlation system may determine phenotypes of a cell dosed with a compound relative to phenotypes of a cell that has not been dosed with a compound. Phenotypes that the high density correlation system may determine, using cell images, include phenotypes related to cell composition, cell death, cell nucleus, morphology, cell mitochondria, cell interactions, cytokines, any suitable phenotype of a cell when a compound is applied to the cell, or a combination thereof. Phenotypes related to cell composition may relate to quantifying T cells, cytotoxic lymphocytes, monocytes, activated monocytes, macrophage, dendritic cells, or any suitable phenotype characterizing a cell composition. Phenotypes related to cell death may relate to quantifying damaged nuclei, dying macrophages, dying T cells, apoptotic cells, or any suitable phenotype characterizing a cell death. Phenotypes related to a cell nucleus may relate to quantifying fragmented nuclei, pyknotic nuclei, kidney-shaped nuclei, or any suitable phenotype characterizing a cell nucleus. Phenotypes related to morphology may relate to quantifying a cell area, maximum radius, mean radius, perimeter, compactness, form factor, or any suitable phenotype characterizing the structure of a cell. Phenotypes related to mitochondria may relate to quantifying T cell reticular mitochondria, T cell fragmentary mitochondria, monocyte reticular mitochondria, monocyte fragmentary mitochondria, or any suitable phenotype characterizing cell mitochondria. Phenotypes related to cell interactions may relate to quantifying lymphocyte-lymphocyte interactions, monocyte-monocyte interactions, lymphocyte-monocyte interactions, or any suitable phenotype characterizing inter-cell interactions. Phenotypes related to cytokines may relate to quantifying the production of interleukin 8 (IL-8), MCP1, IL-6, IL-17A, TNF alpha, IL-4, IL-1β, or any suitable phenotype characterizing cytokines.

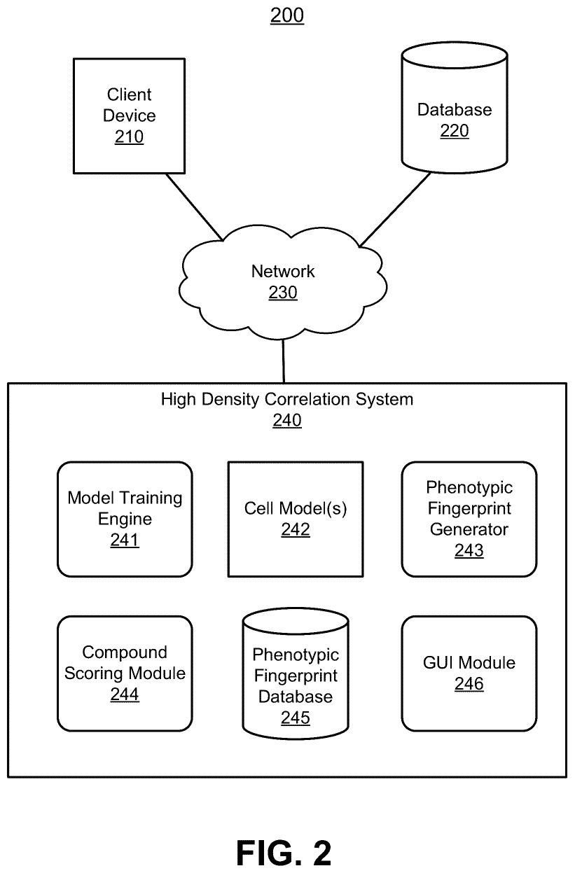

FIG. 2 is a block diagram of a system environment 200 in which a high density correlation system 240 operates, in accordance with at least one embodiment. The system environment 200 includes a client device 210, a database 220, the network 230, and the high density correlation system 240. The high density correlation system 240 may be an embodiment of the high density correlation system 140 of FIG. 1. The system environment 200 may have alternative configurations than shown in FIG. 2, including for example different, fewer, or additional components. For example, the system environment 200 may include one or more laboratory machines that capture data for processing by the high density correlation system 240 or for storage in the database 220.

The client device 210 is a computing device capable of receiving user input as well as transmitting and/or receiving data via the network 230. In some embodiments, the client device 210 is a computer such as a desktop or a laptop computer. Alternatively, the client device 210 may be a device having computer functionality, such as a personal digital assistant (PDA), a mobile telephone, a smartphone, or another suitable device. The client device 210 is configured to communicate with the high density correlation system 240 via the network 230, for example using a native application executed by the client device 210 or through an application programming interface (API) running on a native operating system of the client device 210, such as IOS® or ANDROID™. In another example, the client device 210 is configured to communicate with the high density correlation system 240 via an API running on the high density correlation system 240.

The database 220 stores data for the high density correlation system 240 to determine phenotypic fingerprints. The database 220 may store images of biological samples, which can include images of one or more cells. For example, the database 220 may include images depicting one cell that has been treated or untreated. A treated cell may refer to a cell whose structure, behavior, or other phenotype has been affected by a compound applied to the biological sample containing the cell. Images depicting one cell may be referred to herein as a single cell image. By contrast, an image that depicts two or more cells of a sample may be referred to as a whole view image. The images stored in the database 220 may be labeled images. The labels may indicate one or more phenotypes of a cell or cells depicted in the respective images. For example, the labels may represent a number of IL-8 cytokines produced by the cells depicted. Additionally or alternatively, the labels may indicate a category in which the depicted cell or cells may be categorized in (e.g., as characterized by the one or more phenotypes). For example, the label may include a cell type such as T cell, activated monocyte, or macrophage. In some embodiments, the high density correlation system 240 labels the images and stores them in the database 220. For example, the high density correlation system 240 may use computer vision to determine labels for an unlabeled cell image, apply the determined label, and store the labeled image in the database 220. Alternatively or additionally, the database 220 receives manually labeled cell images.

The database 220 may store data generated by the high density correlation system 240. For example, the database 220 stores phenotypic fingerprints generated by the high density correlation system 240. The database 220 may store proteomics associated with the biological samples, images of which are also processed by the high density correlation system 240. The database 220 may store information regarding known compounds and associated human conditions that the compounds treat. The database 220 may store usage information regarding the high density correlation system 240. This usage information may be anonymized. For example, the database 220 may store usage information indicating frequencies at users query the system 240 for information about particular phenotypes or compounds.

The network 230 serves to communicatively couple the client device 210, the database 220, and the high density correlation system 240. The high density correlation system 240 and the client device 210 are configured to communicate via the network 230, which may comprise any combination of local area and/or wide area networks, using wired and/or wireless communication systems. In some embodiments, the network 230 uses standard communications technologies and/or protocols. For example, the network 230 includes communication links using technologies such as Ethernet, 802.11, worldwide interoperability for microwave access (WiMAX), 3G, 4G, 5G, code division multiple access (CDMA), digital subscriber line (DSL), etc. Examples of networking protocols used for communicating via the network 230 include multiprotocol label switching (MPLS), transmission control protocol/Internet protocol (TCP/IP), hypertext transport protocol (HTTP), simple mail transfer protocol (SMTP), and file transfer protocol (FTP). Data exchanged over the network 230 may be represented using any suitable format, such as hypertext markup language (HTML) or extensible markup language (XML). In some embodiments, all or some of the communication links of the network 230 may be encrypted using any suitable technique or techniques.

The high density correlation system 240 determines phenotypic fingerprints using at least cell images. In some embodiments, the high density correlation system 240 applies machine learning to single cell images to generate phenotypic fingerprints for each single cell image. The high density correlation system 240 may store the generated phenotypic fingerprints in a database for subsequent access in response to a user query. The high density correlation system 240 may receive a user query specifying one or more of a phenotype or a compound, and the relevant phenotypic fingerprints from the database can be returned for display at the user's client device. For example, a user may submit a query to the high density correlation system 240 requesting a list of a particular agonist that increases T cell count and in response, the high density correlation system 240 returns a number of compounds that show a threshold increase in T cell count, where the increase is relative to an amount of T cell counts shown by a vehicle control.

The high density correlation system 240 includes a model training engine 241, one or more cell models 242, a phenotypic fingerprint generator 243, a compound scoring module 244, a phenotypic fingerprint database 245, and a graphic user interface (GUI) module 246. The model training engine 241, the phenotypic fingerprint generator 243, the compound scoring module 244, and the GUI module 246 may be software modules (e.g., code embodied on a machine-readable medium). The high density correlation system 240 may have alternative configurations than shown in FIG. 2, including different, fewer, or additional components. In one example, the high density correlation system 240 may include a preprocessing module for processing cell images before applying a cell model 242 to the cell images (e.g., using an illumination correction function, image segmentation, z-stack processing, etc.). In another example, the high density correlation system 240 may exclude the model training engine 241; rather, the system 240 may receive a cell model that has already been trained external to the system 240.

The model training engine 241 trains machine-learned models of the high density correlation system 240. The model training engine 241 may train one or more of the cell models 242. The model training engine 241 may use one or more of images of cells, proteomics, compound data (e.g., composition of the compound or any suitable data describing the compound, use, or manufacture thereof) of the compound with which the biological sample of cells is dosed, context data related to how the image was captured (e.g., camera sensor, date of capture, etc.), user feedback of the cell model(s) 242, any suitable data for training a model to determine a phenotype of a cell via an image of the cell, or a combination thereof. The images used to train a cell model may be single cell images or whole view images. In one example, the model training engine 241 uses single cell images to train a cell model 242 to determine phenotypes depicted in the single cell images. The images may be of biological samples that have or have not been dosed with a compound (e.g., a drug).

The model training engine 241 may generate training data that includes labeled images. The labels may indicate one or more phenotypes of a cell or cells depicted in the respective images. For example, the labels may represent a cell area of a cell depicted. Additionally or alternatively, the labels may indicate a category in which the depicted cell or cells may be categorized in (e.g., as characterized by the one or more phenotypes). For example, the label may include a cell type such as T cell, activated monocyte, or macrophage. In some embodiments, the model training engine 241 labels the images and stores them in the database 220. For example, the model training engine 241 may use computer vision to determine labels for an unlabeled cell image, apply the determined label, and store the labeled image in the database 220.

In some embodiments, the model training engine 241 can train a machine learning model for determining phenotypes depicted within a cell image in multiple stages. In a first stage, the model training engine 241 may use generalized data collected across various compounds used to dose cells, various physiological profiles of biological sample sources (e.g., different ages, genders, etc.), various human conditions affecting the depicted cells, any suitable characteristic of the cell images, or a combination thereof. For example, the model training engine 241 accesses generalized data of cells dosed with any compound for training a cell model 242 during the first stage, where the training data is labeled to indicate one or more phenotypes exhibited by the depicted cells in the training data. The model training engine 241 may create a first training set based on the labeled generalized data. The model training engine 241 trains a cell model 242, using the first training set, to determine phenotypes and phenotype values exhibited by dosed cells. The determined phenotypes and phenotype values may be structured in a feature vector, which may be referred to herein as an embedding. That is, a cell model 242 is configured to receive, as an input, an image or image data and output an embedding of phenotype values.

In a second stage of training, the model training engine 241 may tailor the phenotype determination of a cell model according to a particular characteristic of cell images and create a second training set using cell images sharing the particular characteristic. For example, during the second stage of training, the model training engine 241 retrains a cell model 242 using images of cells treated with the same compound. Furthermore, the second training set may be created based on user feedback associated with successful or failed phenotype determinations. For example, a user provides feedback that a cell model 242 correctly classified a dying T cell. In response, the model training engine 241 may strengthen a relationship or an association between image data input to the cell model 242 and the phenotype determination by updating the training data using the correct cell death classification (e.g., using the image of the dying T cell applied to the cell model 242 that led to the user feedback).

The model training engine 241 may create a training set including images labeled with a cell type for each cell depicted in the image. For example, the model training engine 241 may label a first set of cell images as depicting activated monocytes, a second set of cell images as depicting T cells, and a third set of cell images as depicting monocytes. The model training engine 241 may then train a model of the cell models 242 to automatically classify a cell type of a cell depicted in an image. The model training engine 241 may apply this model to determine a number of cells and their respective types depicted within an image (e.g., a whole view image). The model may output the location of cells within the image (e.g., image pixel coordinates) and/or bounding boxes around the identified cells. The bounding boxes may be used to extract individual single cell images from a whole view image (e.g., by the phenotypic fingerprint generator 243 for further input of the single view images into the cell model(s) 242).

The cell model(s) 242 may be configured to determine one or more phenotypes and corresponding values depicted in a cell image. The cell model(s) 242 may include machine-learned models, statistical models, or any suitable predictive algorithm for determining a likely phenotype depicted in a cell image. A cell model 242 may be configured to receive, as input, one or more images depicting at least one cell and output a quantitative representation of phenotype values corresponding to phenotypes depicted in the one or more images. The input to the model 242 may also be referred to herein as image data. In some embodiments, the output of a cell model 242 is a feature vector, which may also be referred to as an embedding, with representations of phenotypes serving as dimensions of the feature vector. In one example, each dimension of the feature vector corresponds to a different phenotype and the value of the corresponding phenotype is stored as a feature in that dimension of the embedding. In another example, a single dimension represents two or more phenotypes and the value of the feature can be used (e.g., by the phenotypic fingerprint generator) to derive corresponding values for the two or more phenotypes.

The high density correlation system 240 may include different cell models 242 for different compounds or lack of compound. For example, a first cell model may be used to determine phenotypes of cells that have not been dosed with any compound, a second cell model may be used to determine phenotypes of cells dosed with a first compound, and a third cell model may be used to determine phenotypes of cells dosed with a second compound. In some embodiments, the high density correlation system 240 may include different cell models 242 for identifying different phenotypes. For example, one cell model may be used to determine whether a depicted cell is a monocyte while another cell model may be used to determine whether a depicted cell is a T cell. The cell model(s) 242 may include a model for classifying a cell type of a cell depicted in an image. In some embodiments, the high density correlation system 240 may include different cell models 242 for different types of inputs. For example, one cell model may be configured to determine an embedding from single cell images and another cell model may be configured to determine an embedding from whole view images.

The cell model(s) 242 may use various machine learning techniques such as linear support vector machine (linear SVM), boosting for other algorithms (e.g., AdaBoost), neural networks, logistic regression, naïve Bayes, memory-based learning, random forests, bagged trees, decision trees, boosted trees, boosted stumps, a supervised or unsupervised learning algorithm, or any suitable combination thereof.

The phenotypic fingerprint generator 243 applies a cell model 242 to a cell image of a cell dosed with a compound, receives an embedding representing phenotype values from the cell model 242, and generates a data structure mapping the compound to the phenotype values. This data structure may be referred to herein as a phenotype-compound mapping. One example of a phenotypic fingerprint may be a phenotype-compound mapping. In some embodiments, the phenotypic fingerprint generator 243 curates the input data provided to a cell model 242. For example, the phenotypic fingerprint generator 243 accesses proteomics and high content images from a particular biological sample of PBMC's. In one embodiment, the proteomics may be annotated with timestamps representing the protein data over time (e.g., concentrations of proteins over time). In another embodiment, the proteomics may be annotated with concentration percentages representing the concentration of a particular compound. By analyzing various concentrations of compounds within samples, the high density correlation system 240 may determine a change in phenotypic features of cells as a concentration of a given compound is increased or decreased (e.g., a dose response of the cells). The images may be annotated with timestamps at which they were captured by a camera sensor. The phenotypic fingerprint generator 243 may correlate the timestamps of the proteomics with the corresponding images and input proteomic data and image data having the same or substantially the same (e.g., within a predetermined range of time, such as within ten milliseconds, from one another) timestamp into a cell model 242.

In some embodiments, the phenotypic fingerprint generator 243 may receive, from a cell model, multiple embeddings generated from images of cells dosed with the same compound. The phenotypic fingerprint generator 243 may create a single phenotypic fingerprint from the embeddings by determining an average value for the phenotype values represented in the embedding. In some embodiments, the phenotypic fingerprint generator 243 may additionally or alternatively determine statistical measurements such as a median value or a p-value for the phenotypes. The phenotypic fingerprint generator 243 may store generated phenotypic fingerprints in the database 245. Phenotypic fingerprints stored in the database may be structured for querying (e.g., using a key-value structure) based on a phenotype, compound, or condition). For example, the system 240 may generate 803 the database to map one or more phenotypic fingerprints a compound and map one or more compounds to a condition.

The compound scoring module 244 determines scores for compounds' phenotypic fingerprints. The compound scoring module 244 may use various criteria for scoring the phenotypic fingerprints, including phenotype value, phenotypes known to be correlative to a particular condition, user query data (e.g., frequency of searching for a compound or phenotype) from a single user and/or a population of users, compound characteristics (e.g., compound class or types preferred by the user), toxicity of the compounds, efficacy of the compounds at treating certain conditions, any suitable criterion for scoring a phenotype or compound, or a combination thereof. These criteria may be referred to as phenotype criteria.

The compound scoring module 244 may determine a score for a phenotypic fingerprint based on one or more phenotype criteria. In an example where the compound scoring module 244 uses one phenotype criterion (e.g., a particular phenotype value) to score the phenotypic fingerprints, the compound scoring module 244 may use the phenotype values associated with each compound as corresponding scores (e.g., each phenotypic fingerprint is scored as the number of T cells present).

In another example, the compound scoring module 244 uses two or more phenotype criteria to score the phenotypic fingerprints, where the two or more phenotype criteria may have corresponding weights. The weights may be user specified or automatically determined (e.g., by determining the most frequently used weights by other users). For example, the user may query the high density correlation system 240 for compounds that increase cytokine expression (e.g., an increase in the concentration of TNF alpha) and in response, the compound scoring module 244 may increase the weight corresponding to features of phenotype-compound mappings that characterize a concentration of cytokines. The compound scoring module 244 may then determine scores for a set of phenotype-compound mappings. The compound scoring module 244 may determine which phenotype-compound mappings to score for providing to the user. The compound scoring module 244 may determine to score all available phenotype-compound mappings or subset of available mappings (e.g., by filtering all available mappings). The compound scoring module 244 may filter available mappings based on date of the sample, provider of the sample, type of cell depicted, camera sensor used to capture the image, user feedback received for the sample, any suitable parameter for filtering the images, or a combination thereof.

In some embodiments, the compound scoring module 244 may automatically score phenotypic fingerprints in the phenotypic fingerprint database 245. For example, the compound scoring module 244 may score phenotypic fingerprints in response to an update of the database 245 with a new phenotypic fingerprint, removal of a phenotypic fingerprint, or updating of an existing compound's phenotypic fingerprint. In another example, the compound scoring module 244 may score the phenotypic fingerprints periodically (e.g., every week, every month, etc.). In some embodiments, the compound scoring module 244 may determine a subset of the phenotypic fingerprints in the phenotypic fingerprint database 245 to score. For example, the compound scoring module 244 may determine a subset of known compounds used to treat a particular condition and then score the determined subset. That is, the high density correlation system 240 may automatically score phenotypic fingerprints based on determined correlations (e.g., between conditions and compounds, between compounds, between phenotypes, etc.). The high density correlation system 240 may present the automatically scored compounds for display at a GUI without necessarily receiving a user request to display the scored compounds. For example, the high density correlation system 240 may display the automatically scored compounds as an initial or default arrangement of phenotypic fingerprints in a GUI before a user has specified an input at the GUI for scoring the compounds.

The compound scoring module 244 may determine a correlation between phenotypic fingerprints. The compound scoring module 244 may score phenotypic fingerprints according to an amount of correlation (e.g., phenotypic fingerprints more similar to a target phenotypic fingerprint are scored higher than phenotypic fingerprints that are less similar to the target phenotypic fingerprint). In one example, the compound scoring module 244 may determine, using phenotypic fingerprints of compounds stored in the phenotypic fingerprint database 245, that two compounds that treat different human conditions show a similar percentage of damaged nuclei. In another example, the compound scoring module 244 may determine that two different compounds affect macrophages similarly and can be substituted for one another. In some embodiments, the compound scoring module 244 may determine and rank phenotypes correlative to a human condition. For example, the compound scoring module 244 may access phenotypic fingerprints of cells that have not been dosed with a compound and are associated with a particular condition. The compound scoring module 244 may then compare the phenotypic fingerprints to one another to determine a correlation between the particular condition and similarly exhibited phenotypes throughout images of cells affected by the condition.

The phenotypic fingerprint database 245 stores phenotypic fingerprints of cells. The phenotypic fingerprints may be generated by the phenotypic fingerprint generator 243. The cells may be treated with a compound or untreated. The phenotypic fingerprints may be stored with data about the biological sample from which the phenotypic fingerprint was determined. The data about the biological sample may include one or more conditions (e.g., psoriasis) affecting the source of the biological sample. The phenotypic fingerprints within the phenotypic fingerprint database 245 may be accessed by the compound scoring module 244 to score and/or rank the phenotypic fingerprints or determine correlations between the phenotypic fingerprints. The phenotypic fingerprints within the phenotypic fingerprint database 245 may be accessed by the GUI module 246 for displaying at the client device 210 (e.g., graphics depicting phenotypes of treated cells compared to untreated cells).

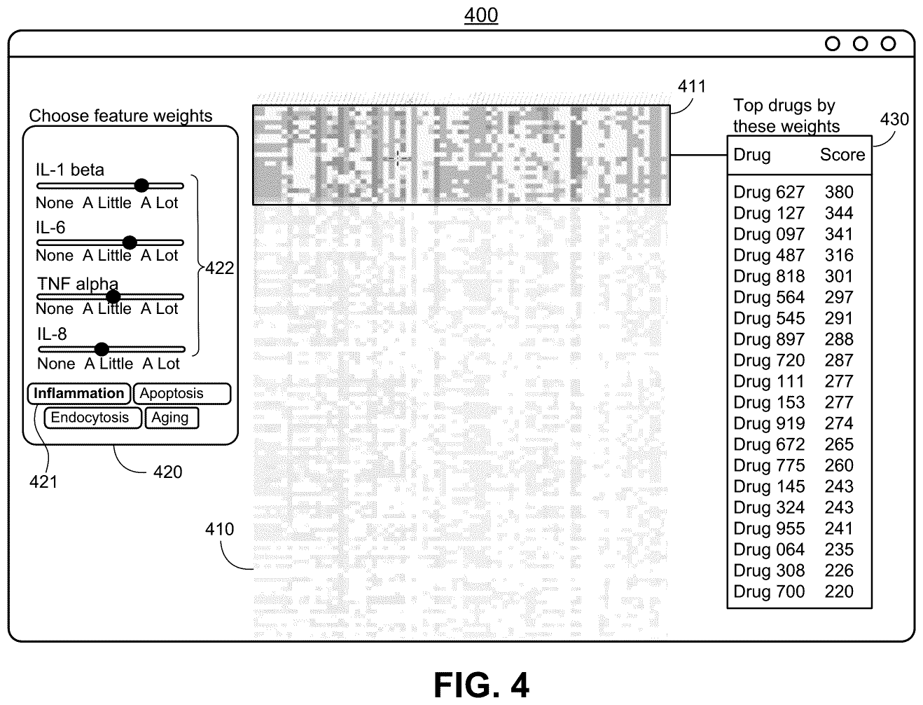

The GUI module 246 generates one or more GUIs for display at a client device (e.g., the client device 210). The generated GUI may be interactive, including user inputs for querying the high density correlation system 240 for information regarding a phenotype, compound, condition, or effect of a compound on a condition. The GUI module 246 may use the user query to instruct the compound scoring module 244 to score the phenotypic fingerprints. The GUI module 246 may display the scored phenotypic fingerprints (e.g., as shown in FIG. 4). Examples of GUIs that may be generated by the GUI module 246 are described with respect to FIGS. 4-7.

In some embodiments, the GUI module 246 includes an interface for client devices to communicate with the high density correlation system 240. For example, the GUI module 246 may include an API for clients of the high density correlation system 240 to retrieve data stored in the phenotypic fingerprint database 245, send query requests, and make settings through a programming language. Various functionalities of the software modules of the high density correlation system 240, such as the scoring algorithm applied by the compound scoring module 244, may be changed by the clients through sending commands to the API.

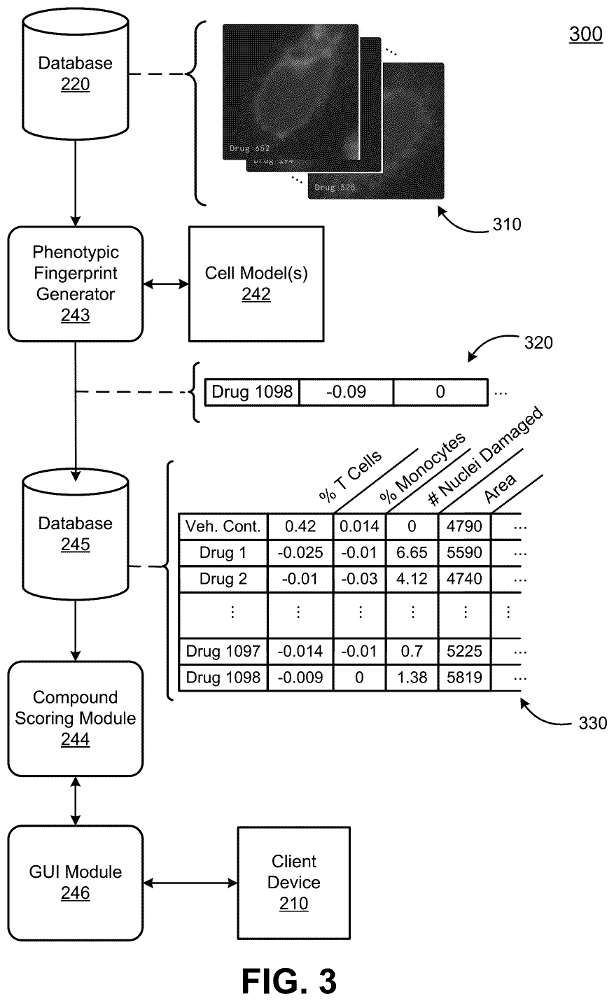

FIG. 3 shows a block diagram of a process 300 for providing a visualization of phenotypic fingerprints of a biological sample, in accordance with one embodiment. Components of the high density correlation system 240 can perform the process 300. In some embodiments, additional or fewer operations may be performed than shown in the process 300. For example, the high density correlation system 240 may preprocess images of cells received from the database 220 (e.g., if the images have not already been preprocessed).

In the process 300, the phenotypic fingerprint generator 243 receives single cell images 310 from the database 220. The single cell images 310 may be from a biological sample of PBMC's treated with a compound, drug 1098. The phenotypic fingerprint generator 243 applies the cell model(s) 242 to the single cell images 310. In some embodiments, the phenotypic fingerprint generator 243 may apply different cell models to determine different phenotypes depicted in the single cell images 310. For example, the phenotypic fingerprint generator 243 may apply a first cell model for determining phenotypes related to cell composition to all of the single cell images 310, apply a second cell model for determining phenotypes related to cell death to all of the single cell images 310, and apply additional cell models for different categories of phenotypes to receive, as an output from the cell models, various identified phenotypes and corresponding values. In some embodiments, the phenotypic fingerprint generator 243 may apply different cell models 242 based on a cell type of cells depicted in the single cell images 310. For example, the phenotypic fingerprint generator 243 may first apply one of the cell models 242 to the single cell images 310 that classifies each cell depicted into a particular type of cell (e.g., T cell, macrophage, etc.). The phenotypic fingerprint generator 243 may then apply other cell models of the models 242, where each of the other cell models determines various phenotypes for a particular cell type.

After receiving an embedding output from the cell model(s) 242, the phenotypic fingerprint generator 243 generates a phenotype-compound mapping 320 that serves as a phenotypic fingerprint representing the effect of the compound, drug 1098, on the cells depicted in the single cell images 310. In some embodiments, a subset of the values of the embedding may represent the value of the phenotype relative to the value as determined from images of untreated cells (e.g., a vehicle control). The phenotypic fingerprint generator 243 stores the mapping 320 into the database 245, where the phenotype-compound mappings may be arranged in a data structure 330 of compounds and phenotypes.

The compound scoring module 245 can score phenotypic fingerprints in the database 245 based on a user query received from the client device 210 via a GUI generated by the GUI module 246. FIGS. 4, 6, and 7 show example GUI's with user inputs for querying for information from the high density correlation system 240. The user query may request a list of compounds that cause cells to exhibit a particular phenotype (e.g., compounds that increase T cell count). The GUI module 246 can instruct the compound scoring module 244 to score the phenotypic fingerprints in the database 245 to determine a subset of compounds that increase T cell count relative to other compounds.

### Example GUIs of the High Density Correlation System

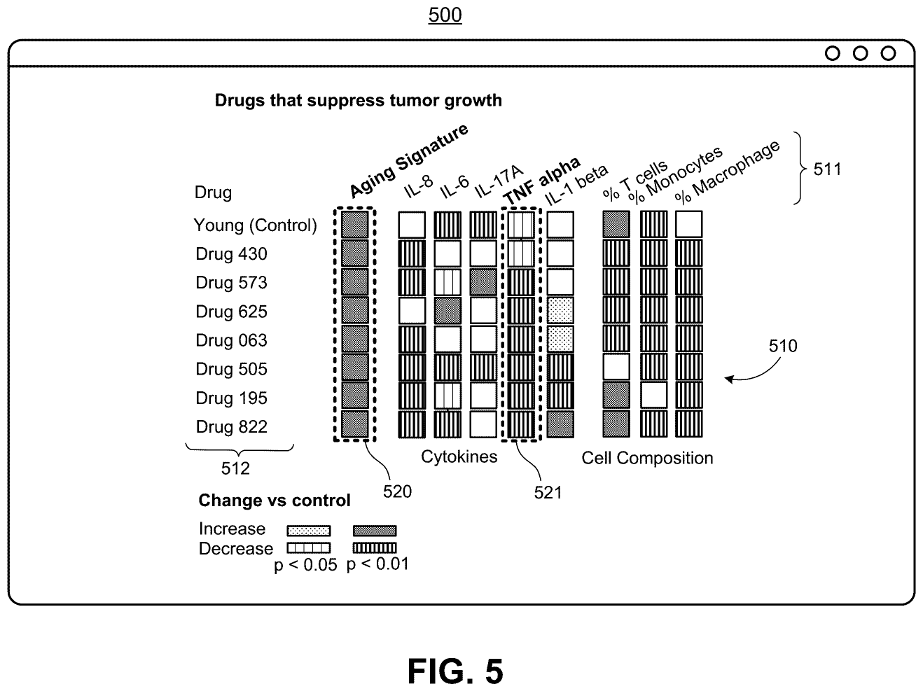

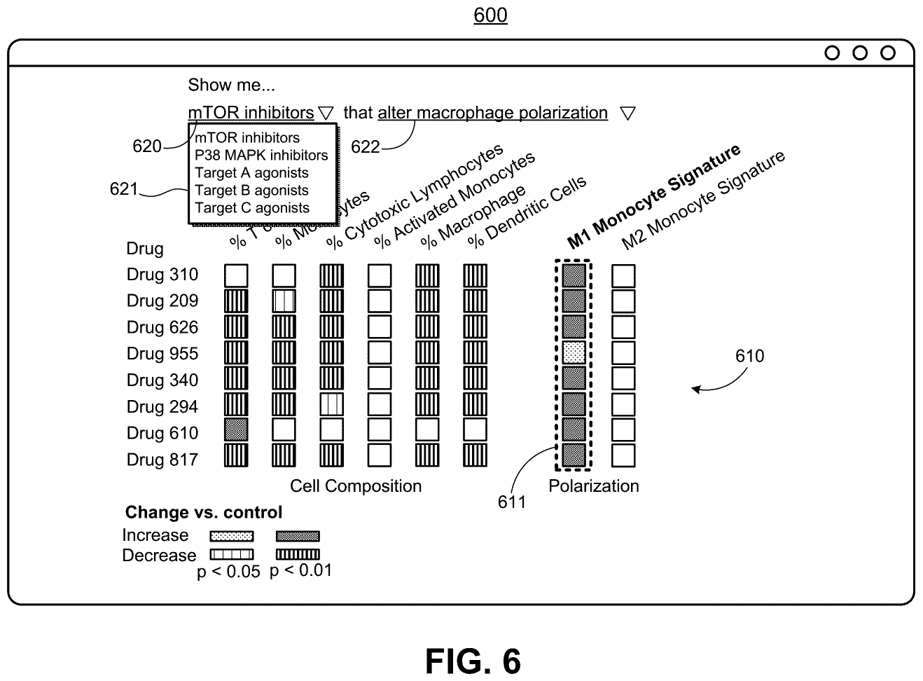

FIG. 4 shows a GUI 400 for identifying compounds with customized scoring, in accordance with at least one embodiment. The GUI 400 may be generated by the GUI module 246 of the high density correlation system 240. The GUI 400 includes a color-coded chart 410 of phenotype values. Each row of the chart 410 may represent a phenotypic fingerprint of a compound and each column may represent a phenotype. Each square or block within the chart 410 can represent a statistical significance comparison between treatment and control conditions. The colors of the chart 410 may represent a magnitude of a p-value associated with the measured phenotype value. Additionally or alternatively, the colors of the chart 410 may represent a level of increase, a level of decrease, or lack of change of the phenotype's value for a cell treated by a particular compound relative to the phenotype's value for untreated cells. For example, a first color may represent an increase in a mean value of a phenotype and a second color may represent a decrease in the mean value of the phenotype. Furthermore, a light and a dark shade of each color may be used to represent a p-value less than 0.05 and a p-value greater than 0.01, respectively. FIGS. 5 and 6 show magnified examples of color-coded charts.

Overlayed on the chart 410 is a frame 411 that visually distinguishes a subset of phenotypic fingerprints displayed in the chart 410. In particular, the frame 411 visually distinguishes the compounds with the highest scores according to user specified weights from other compounds (e.g., by lowering the intensity or brightness of the colors representing lower scored phenotypic fingerprints outside of the frame 411). In some embodiments, the GUI module 246 may monitor a position of a user cursor at the GUI 400 and determine to show additional information regarding a phenotype in response to determining that the user's cursor is hovering over the phenotype in the chart 410.

A user may input weights in a weight selection interface 420. The weight selection interface 240 includes user inputs 421 for selecting a condition and user inputs 422 for selecting weights. The user may select one of the user inputs 421 and in response, the GUI module 246 may update the GUI 400 to show different weights as the user inputs 422. For example, while the condition of inflammation may correspond to the weights depicted in FIG. 4, the weights for apoptosis may be a different set of phenotypes. Accordingly, a user selection of the user inputs 421 may cause the GUI module 246 to update the GUI 400 to display a different set of weights for user selection among the user inputs 422. Determination of which phenotypes are displayed among the user inputs 422 is described with respect to the compound scoring module 244 in the description of FIG. 2. The GUI module 246 may receive a user selection of one or more of the weights among the user inputs 422. Although shown as a slider tool, the GUI module 246 may use any suitable form of user input for specifying a quantity of weight a user requests to place on a particular phenotype.

A result table 430 is displayed in the GUI 400. The table 430 includes a sorted list of compounds. The list of compounds may be scored based on the weights specified through the inputs 422. The list of compounds included in the table 430 can correspond to the compounds within the frame 411.

FIG. 5 shows a GUI 500 for identifying compounds that correlate with an effect on a condition, in accordance with at least one embodiment. The GUI 500 may be generated by the GUI module 246. The GUI 500 shows a color-coded chart 510 of phenotypic fingerprints for compounds 512 causing cells to exhibit phenotypes 511 when dosed with the compounds 512. In particular, the GUI 500 depicts a list of compounds 512 that suppress tumor growth. The high density correlation system 240 may determine, using determined correlations between phenotypes and known compounds that suppress tumor growth, to score phenotypic fingerprints by applying greater weights to phenotypes such as the aging signature and TNF alpha concentration to determine the compounds 512. After scoring the phenotypic fingerprints for various compounds using the weights, the high density correlation system 240 may determine to display a subset of the various compounds (i.e., the compounds 512). Using the GUI 500, the high density correlation system 240 can demonstrate that the compounds 512 exhibit the similar aging signature phenotype 520 and TNF alpha concentration phenotype 521. That is, the compounds 512 show a similar increase in aging signature and decrease in concentration of TNF alpha.

The GUI 500 may be displayed in response to a user query. An example of a similar interface with user inputs for specifying a query is shown in FIG. 6. Additionally, the number of columns or phenotypes shown may be greater than what is depicted in FIG. 6 (e.g., as many columns as depicted in FIG. 4 may be depicted). In some embodiments, the high density correlation system 240 may limit the number phenotypes shown. For example, the high density correlation system 240 may limit the phenotypes presented to the phenotypes having the greatest relevancy to a particular condition or effect on the condition (e.g., suppressing tumor growth). Accordingly, the high density correlation system 240 may further simplify a view of high density cell image and proteomics data and correlation from a large, color-coded chart as shown in FIG. 4 to a smaller, color-coded chart as shown in FIGS. 5 and 6.

FIG. 6 shows a GUI 600 for querying a high density correlation system, in accordance with at least one embodiment. The GUI 600 may be generated by the GUI module 246. The GUI 600 shows a color-coded chart 610 showing compounds that produced changes in macrophage polarization as demonstrated by increases in the polarization of the M1 monocyte signature phenotype 611. The results shown in the chart 610 may be displayed in response to a user query requesting that the high density correlation system 420 show mTOR inhibitors that alter macrophage polarization. The user query may be input through input fields 620 and 622 enabling the user to specify a type or class of compound and to specify a condition or an effect on a condition, respectively. The input fields 620 and 622 may be dropdown menus, as shown by the dropdown menu 621. Alternatively, the input fields 620 and 622 may be any suitable form of input field for specifying a string indicating a compound or condition. The dropdown menu 621 shows an example of a list of possible compounds that can be selected by the user for querying the high density correlation system 240. In other embodiments, the dropdown menu may include additional, different, or fewer compounds. In some embodiments, the GUI module 246 may determine which compounds to display in an input field of preselected compounds, conditions, or effects based on usage information. For example, the GUI module 246 may prepopulate dropdown menu 621 with a list of compounds determined to be the most frequently queried compounds by users of the high density correlation system 240. In another example, the GUI module 246 may track previously queried information and prepopulate input fields with a list of recently queried information (e.g., the five most recently queried compounds by the user).

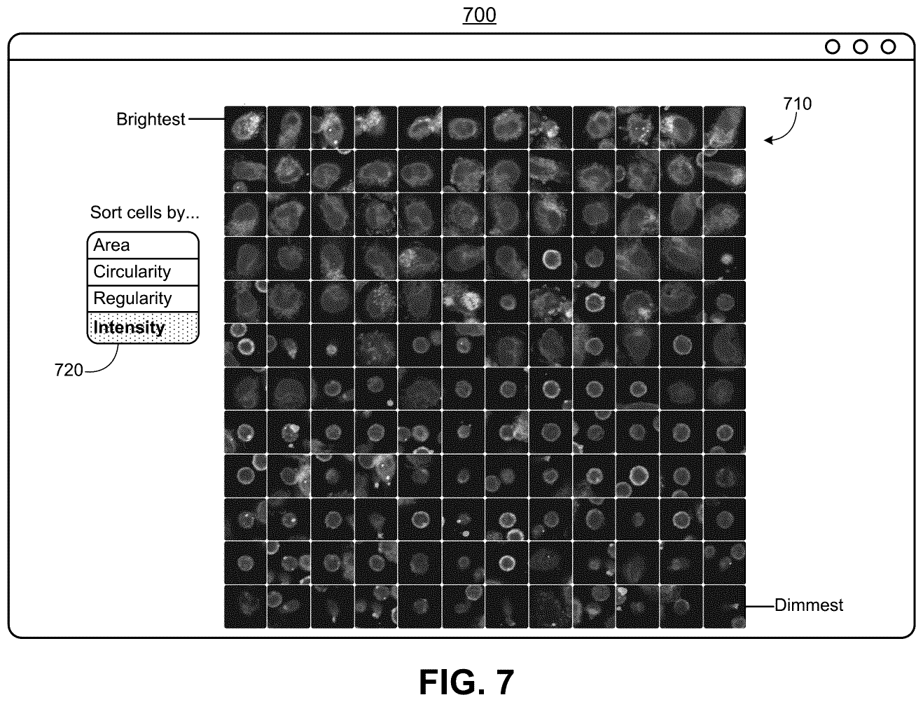

FIG. 7 shows a GUI 700 for visualizing profiles of cells, in accordance with at least one embodiment. The GUI 700 may be generated by the GUI module 246. The GUI 700 includes a grid 710 of single cell images. An image that depicts multiple cells that is processed by the high density correlation system 240 to focus on a single cell in the image may still be referred to herein as a single cell image. The GUI module 246 may determine to sort the images in the grid 710 based on a phenotype exhibited by the cells depicted. The phenotype may be selected by a user via a sorting menu 720 of various phenotypes. The GUI 700 is depicted with the phenotype of intensity being selected, where the images are sorted from brightest to dimmest cell.

### Example Process Using the High Density Correlation System

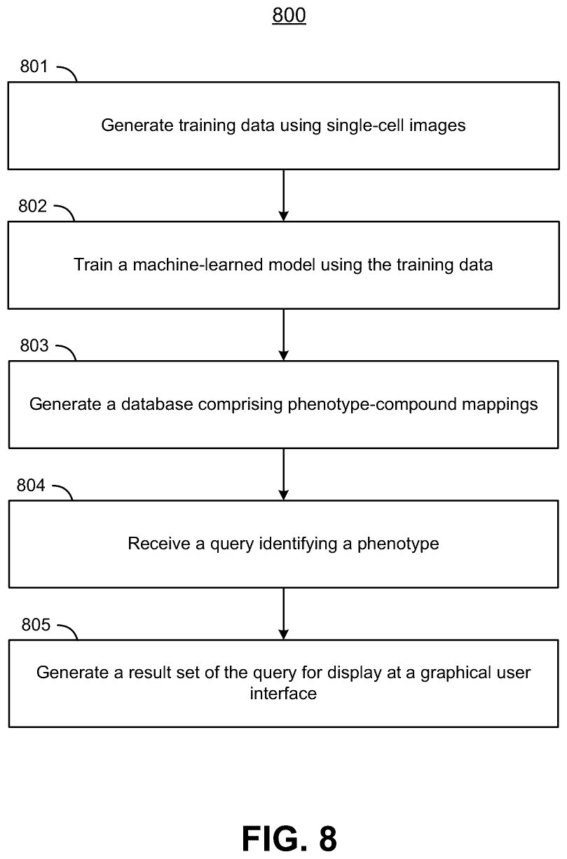

FIG. 8 is a flowchart illustrating a process 800 for identifying phenotypic fingerprints using the high density correlation system described herein, in accordance with at least one embodiment. The process 800 may be performed by the high density correlation system 240. However, some or all of the operations may be performed by other entities or components. In addition, some embodiments may perform the operations in parallel, perform the operations in different orders, or perform different operations. For example, although FIG. 8 shows the use of single cell images to determine phenotype-compound mappings, the high density correlation system 240 may use whole view images in addition or alternatively. In another example, the high density correlation system 240 may receive a trained model, omitting the operations in the process 800 for generating training data and training a model.

The high density correlation system 240 generates 801 training data using single-cell images. The system 240 may generate 801 training data using images of untreated cells and treated cells of a biological sample associated with a condition (e.g., an inflammatory disease). The images of treated cells may include images of cells treated with respective compounds. The system 240 may label the images with respective labels representing the phenotypes and corresponding phenotype values depicted in each image. The labeled images may be included within the generated 801 training data. In a first example, the system 240 generates 801 training data using images of cells from persons being treated with compounds to address their noncancerous tumors, where the training data includes images labeled with labels representing phenotypes exhibited by the cells. In a second example, the system 240 generates 801 training data using images of cells from persons being vaccinated and treated with compounds that improve their vaccine response. In a third example, the system 240 generates 801 training data using images of cells from persons being treated with compounds to address inflammatory diseases (e.g., rheumatoid arthritis).

The high density correlation system 240 trains 802 a machine-learned model using the training data. The trained machine-learned model may be configured to determine, based on an image of a cell, one or more phenotypes of the cell having a compound applied to the cell. Additional information on training a cell model is described with reference to the model training engine 241 in the description of FIG. 2. In the first example, the system 240 trains 802 a machine-learned model using the training data of cells dosed with a compound associated with treating non-cancerous tumors, where the trained model may identify phenotypes in cell images similar to the phenotypes exhibited by the treated cells. In the second example, the system 240 trains 802 a machine-learned model using the training data of cells dosed with a compound associated with increasing vaccine response, where the trained model may identify phenotypes that are similar to the phenotypes exhibited by the treated cells. In the third example, the system 240 trains 802 a machine-learned model using the training data of cells dosed with a compound associated with treating an inflammatory disease, where the trained model may identify phenotypes that are similar to the phenotypes exhibited by the treated cells.

The high density correlation system 240 generates 803 a database comprising phenotype-compound mappings. The system 240 may use the outputs of the trained 802 machine-learned model to generate 803 the database (e.g., the phenotypic fingerprint database 245). The database may include data structures of phenotype-compound mappings, an example of which is depicted in FIG. 3. In the first example, the system 240 generates 803 a database of phenotypic fingerprints associated with compounds for treating noncancerous tumors. In the second example, the system 240 generates 803 a database of phenotypic fingerprints associated with compounds that increase vaccine response. In the third example, the system 240 generates 803 a database of phenotypic fingerprints associated with compounds that treat inflammatory diseases. In some embodiments, the phenotypic fingerprints generated in these three examples may be stored in the same database.

The high density correlation system 240 receives 804 a query identifying a phenotype. In the first example, the system 240 receives 804 a query for compounds that suppress tumor growth. The system 240 may determine, using a correlation between tumor suppression and phenotypes represented in the stored phenotypic fingerprints, that the query identifies one or more phenotypes associated with suppressing tumor growth. In the second example, the system 240 receives 804 a query for compounds that improve vaccine response. The system 240 may determine, using a correlation between vaccine response and phenotypes represented in the stored phenotypic fingerprints, that the query identifies one or more phenotypes associated with increased vaccine response. In the third example, the system 240 receives 804 a query for compounds that inhibit inflammation. The system 240 may determine, using a correlation between compounds for treating inflammatory diseases and phenotypes represented in the stored phenotypic fingerprints, that the query identifies one or more phenotypes associated with inhibiting inflammation.

The high density correlation system 240 generates 805 a result set of the query for display at a GUI. The result set may identify compounds corresponding to the identified phenotype. Furthermore, the compounds can be ordered based on a score for each compound. In the first example, the system 240 generates 805 a result set of compounds that suppress tumor growth (e.g., as shown in FIG. 5). The generated result set may show the values of the identified phenotypes for suppressing tumor growth (e.g., an aging signature an amount of TNF alpha cytokine). In the second example, the system 240 generates 805 a result set of compounds that improve vaccine response. The generated result set may show activator signatures, IL-6 concentration, and monocyte fragmentary mitochondria concentration as some of the identified phenotypes. In the third example, the system 240 generates 805 a result of compounds that inhibit inflammation. The generated result set may show a signature of a particular inhibitor and an IL-1β concentration as some of the identified phenotypes.

### ADDITIONAL CONSIDERATIONS

The foregoing description of the embodiments has been presented for the purpose of illustration; it is not intended to be exhaustive or to limit the patent rights to the precise forms disclosed. Persons skilled in the relevant art can appreciate that many modifications and variations are possible in light of the above disclosure.

Some portions of this description describe the embodiments in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. In one embodiment, a software module is implemented with a computer program product comprising a computer-readable medium containing computer program code, which can be executed by a computer processor for performing any or all of the steps, operations, or processes described.

Embodiments may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a non-transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

Embodiments may also relate to a product that is produced by a computing process described herein. Such a product may comprise information resulting from a computing process, where the information is stored on a non-transitory, tangible computer readable storage medium and may include any embodiment of a computer program product or other data combination described herein.

Finally, the language used in the specification has been principally selected for readability and instructional purposes, and it may not have been selected to delineate or circumscribe the patent rights. It is therefore intended that the scope of the patent rights be limited not by this detailed description, but rather by any claims that issue on an application based hereon. Accordingly, the disclosure of the embodiments is intended to be illustrative, but not limiting, of the scope of the patent rights, which is set forth in the following claims.

What is claimed is:

1. A method comprising:
generating training data using single-cell images;
training a machine-learned model using the training data, the machine-learned model configured to determine one or more phenotypes of a cell having a compound applied to the cell based on an image of the cell;
generating a database comprising phenotype-compound mappings generated based on outputs of the machine-learned model;
receiving a query identifying a phenotype; and
generating a result set of the query using the database for display at a graphical user interface (GUI), the result set identifying compounds corresponding to the identified phenotype, the compounds ordered based on one or more phenotype criteria.

2. The method of claim 1, wherein generating the training data comprises:
receiving the single-cell images of a sample including peripheral blood mononuclear cells, the images captured by an image sensor;
processing the single-cell images using one or more of illumination correction, segmentation, or z-stack processing; and
labeling the single-cell images using one or more labels indicating respective phenotypes depicted in the single-cell images.

3. The method of claim 1, further comprising scoring the identified compounds using the one or more phenotype criteria, wherein scoring the identified compounds comprises:
generating visual indicators of conditions for display at the GUI, each condition associated with a combination of phenotypes corresponding to a phenotype criterion;
in response to receiving a selection of a visual indicator of a condition, updating the GUI to display input fields for weights of the corresponding combination of phenotypes associated with the condition; and
applying one or more weights to respective phenotypes associated with the identified compounds, the one or more weights received via the input fields.

4. The method of claim 3, further comprising:
determining phenotypic correlations between known compounds and the conditions; and
determining the combination of phenotypes for each condition based on the determined phenotypic correlations.

5. The method of claim 1, wherein the single-cell images are a first set of single-cell images and wherein the compound is a first compound, further comprising:
applying the machine-learned model to a second set of single-cell images depicting one or more cells having a second compound applied to the one or more cells; and
receiving a plurality of embeddings as output from the machine-learned model, the plurality of embeddings corresponding to phenotypic characteristics represented in the second set of single-cell images.

6. The method of claim 5, wherein the phenotype-compound mappings are a first set of phenotype-compound mappings, further comprising:
generating a second set of phenotype-compound mappings mapping each of the plurality of embeddings to the second compound; and
updating the database to further include the second set of phenotype-compound mappings, wherein the first set of phenotype-compound mappings is associated with the first compound.

7. The method of claim 1, wherein the single-cell images are a first set of single-cell images, further comprising:
generating a second set of single-cell images for display at the GUI, the second set of single-cell images generated for display in a first order, wherein each of the second set of single-cell images is associated with a phenotype-compound mapping generated by the machine-learned model;
generating a sorting menu for display at the GUI, the sorting menu comprising a list of phenotypic characteristics represented in the phenotype-compound mappings associated with the second set of single-cell images; and
in response to receiving a selection of a phenotypic characteristic in the list:
determining a second order based on the selected phenotypic characteristic and values of the selected phenotypic characteristic in each of the phenotype-compound mappings associated with the second set of single-cell images; and
updating the second set of single-cell images for display in a second order at the GUI.

8. The method of claim 1, wherein the single-cell images are a first set of single-cell images, further comprising:
classifying a second set of single-cell images using the machine-learned model, the second set of single-cell images extracted from a whole-view image of a sample comprising single cells depicted in the second set of single-cell images, the second set of single-cell images classified based on a plurality of cell types of the single cells;
generating the classified second set of single-cell images grouped for display at the GUI based on the plurality of cell types; and
generating the whole-view image of the sample and a plurality of visual indicators of each of the classified second set of single-cell images within the whole-view image for display at the GUI.

9. The method of claim 1, wherein the single-cell images are a first set of single-cell images, further comprising:
identifying a cell type depicted in the first set of single-cell images;
receiving a second set of single-cell images of cells having the compound applied to the cells and having the cell type; and
re-training the machine-learned model using the second set of single-cell images.

10. A non-transitory computer readable medium comprising stored instructions that, when executed by one or more processors, cause the one or more processors to:
generate training data using single-cell images;
train a machine-learned model using the training data, the machine-learned model configured to determine one or more phenotypes of a cell having a compound applied to the cell based on an image of the cell;
generate a database comprising phenotype-compound mappings generated based on outputs of the machine-learned model;
receive a query identifying a phenotype; and
generate a result set of the query using the database for display at a graphical user interface (GUI), the result set identifying compounds corresponding to the identified phenotype, the compounds ordered based on a score for each compound.

11. The non-transitory computer readable medium of claim 10, wherein the instructions, when executed by the one or more processors, further cause the one or more processors to:
receive the single-cell images of a sample including peripheral blood mononuclear cells, the images captured by an image sensor;
process the single-cell images using one or more of illumination correction, segmentation, or z-stack processing; and
label the single-cell images using one or more labels indicating respective phenotypes depicted in the single-cell images.

12. The non-transitory computer readable medium of claim 10, wherein the instructions, when executed by the one or more processors, further cause the one or more processors to score the identified compounds using the one or more phenotype criteria by:
generating visual indicators of conditions for display at the GUI, each condition associated with a combination of phenotypes corresponding to a phenotype criterion;
in response to receiving a selection of a visual indicator of a condition, updating the GUI to display input fields for weights of the corresponding combination of phenotypes associated with the condition; and
applying one or more weights to respective phenotypes associated with the identified compounds, the one or more weights received via the input fields.

13. The non-transitory computer readable medium of claim 12, wherein the instructions, when executed by the one or more processors, further cause the one or more processors to:
determine phenotypic correlations between known compounds and the conditions; and
determine the combination of phenotypes for each condition based on the determined phenotypic correlations.

14. The non-transitory computer readable medium of claim 10, wherein the single-cell images are a first set of single-cell images, and wherein the instructions, when executed by the one or more processors, further cause the one or more processors to:
generate a second set of single-cell images for display at the GUI, the second set of single-cell images generated for display in a first order, wherein each of the second set of single-cell images is associated with a phenotype-compound mapping generated by the machine-learned model;
generate a sorting menu for display at the GUI, the sorting menu comprising a list of phenotypic characteristics represented in the phenotype-compound mappings associated with the second set of single-cell images; and
in response to receiving a selection of a phenotypic characteristic in the list:
determine a second order based on the selected phenotypic characteristic and values of the selected phenotypic characteristic in each of the phenotype-compound mappings associated with the second set of single-cell images; and
update the second set of single-cell images for display in a second order at the GUI.

15. The non-transitory computer readable medium of claim 10, wherein the single-cell images are a first set of single-cell images, and wherein the instructions, when executed by the one or more processors, further cause the one or more processors to:
classify a second set of single-cell images using the machine-learned model, the second set of single-cell images extracted from a whole-view image of a sample comprising single cells depicted in the second set of single-cell images, the second set of single-cell images classified based on a plurality of cell types of the single cells;
generate the classified second set of single-cell images grouped for display at the GUI based on the plurality of cell types; and
generate the whole-view image of the sample and a plurality of visual indicators of each of the classified second set of single-cell images within the whole-view image for display at the GUI.

16. A system comprising:
one or more processors; and
a non-transitory computer readable storage medium storing executable instructions that, when executed by one or more processors, cause the one or more processors to:
generate training data using single-cell images;
train a machine-learned model using the training data, the machine-learned model configured to determine one or more phenotypes of a cell having a compound applied to the cell based on an image of the cell;
generate a database comprising phenotype-compound mappings generated based on outputs of the machine-learned model;
receive a query identifying a phenotype; and
generate a result set of the query using the database for display at a graphical user interface (GUI), the result set identifying compounds corresponding to the identified phenotype, the compounds ordered based on a score for each compound.

17. The system of claim 16, wherein the instructions, when executed by the one or more processors, further cause the one or more processors to:
receive the single-cell images of a sample including peripheral blood mononuclear cells, the images captured by an image sensor;
process the single-cell images using one or more of illumination correction, segmentation, or z-stack processing; and
label the single-cell images using one or more labels indicating respective phenotypes depicted in the single-cell images.

18. The system of claim 16, wherein the instructions, when executed by the one or more processors, further cause the one or more processors to score the identified compounds using the one or more phenotype criteria by:
generating visual indicators of conditions for display at the GUI, each condition associated with a combination of phenotypes corresponding to a phenotype criterion;
in response to receiving a selection of a visual indicator of a condition, updating the GUI to display input fields for weights of the corresponding combination of phenotypes associated with the condition; and
applying one or more weights to respective phenotypes associated with the identified compounds, the one or more weights received via the input fields.

19. The system of claim 16, wherein the single-cell images are a first set of single-cell images, and wherein the instructions, when executed by the one or more processors, further cause the one or more processors to:
generate a second set of single-cell images for display at the GUI, the second set of single-cell images generated for display in a first order, wherein each of the second set of single-cell images is associated with a phenotype-compound mapping generated by the machine-learned model;
generate a sorting menu for display at the GUI, the sorting menu comprising a list of phenotypic characteristics represented in the phenotype-compound mappings associated with the second set of single-cell images; and
in response to receiving a selection of a phenotypic characteristic in the list:
determine a second order based on the selected phenotypic characteristic and values of the selected phenotypic characteristic in each of the phenotype-compound mappings associated with the second set of single-cell images; and
update the second set of single-cell images for display in a second order at the GUI.

20. The system of claim 16, wherein the single-cell images are a first set of single-cell images, and wherein the instructions, when executed by the one or more processors, further cause the one or more processors to:
classify a second set of single-cell images using the machine-learned model, the second set of single-cell images extracted from a whole-view image of a sample comprising single cells depicted in the second set of single-cell images, the second set of single-cell images classified based on a plurality of cell types of the single cells;
generate the classified second set of single-cell images grouped for display at the GUI based on the plurality of cell types; and
generate the whole-view image of the sample and a plurality of visual indicators of each of the classified second set of single-cell images within the whole-view image for display at the GUI.
